SEQUENCE LISTING (5 sequences)

SEQ ID NO: 1 - protein, 39 aa, Artificial Sequence
  Synthetic construct
HGEGTFSSELATILDALAARDFIAWLIATKITDKKKKKK

SEQ ID NO: 2 - protein, 34 aa, Artificial Sequence
  Synthetic construct
HGEGTFSSELATILDALAARDFIAWLIATKITDK

SEQ ID NO: 3 - protein, 35 aa, Artificial Sequence
  Synthetic construct
HGEGTFSSELATILDALAARDFIAWLIATKITDKK

SEQ ID NO: 4 - protein, 33 aa, Artificial Sequence
  Synthetic construct
HGDGSFSDEMNTILDNLAARDFINWLIQTKITD

SEQ ID NO: 5 - protein, 33 aa, Artificial Sequence
  Synthetic construct
HADGSFSDEMNTILDNLAARDFINWLIQTKITD

(19) United States
(12) Patent Application Publication
(10) Pub. No.: US 2024/0091317 A1
(43) Pub. Date: Mar. 21, 2024

(54) USE OF GLP-2 ANALOGUES IN PATIENTS WITH RENAL INSUFFICIENCY

(71) Applicant: Zealand Pharma A/S, Søborg (DK)

(72) Inventors: Kim SONNE, Søborg (DK); Mikkel Askjær AGERSNAP, Søborg (DK); Kim Mark KNUDSEN, Søborg (DK)

(21) Appl. No.: 18/268,139

(22) PCT Filed: Dec. 15, 2021
(86) PCT No.: PCT/EP2021/085846

§ 371 Date: Jun. 16, 2023

(30) Foreign Application Priority Data
Dec. 16, 2020 (EP) 20214763.3
Mar. 30, 2021 (EP) 21166078.2

(51) Int. Cl. A61K 38/26

(52) U.S. Cl. CPC A61K 38/26

(57) ABSTRACT

The present invention relates to dosage regimes for the administration of glucagon-like-peptide-2 (GLP-2) analogues, and in particular to ZP1848 (glepaglutide), in patients with renal insufficiency. Unlike other GLP-2 analogues, such as teduglutide, it has been found that ZP1848 is cleared normally in patients with renal impairment. As a result, there is no need to adjust the dosage of ZP1848 depending on a patient's renal function.

### FIELD OF THE INVENTION

The present invention relates to dosage regimes for the administration of glucagon-like-peptide-2 (GLP-2) analogues, and in particular to ZP1848 (glepaglutide), in patients with renal insufficiency.

### BACKGROUND OF THE INVENTION

Human GLP-2 is a 33-amino-acid peptide with the following sequence: Hy-His-Ala-Asp-Gly-Ser-Phe-Ser-Asp-Glu-Met-Asn-Thr-Ile-Leu-Asp-Asn-Leu-Ala-Ala-Arg-Asp-Phe-Ile-Asn-Trp-Leu-Ile-Gln-Thr-Lys-Ile-Thr-Asp-OH. It is derived from specific post-translational processing of proglucagon in the enteroendocrine L cells of the intestine and in specific regions of the brainstem. GLP-2 binds to a single G-protein-coupled receptor belonging to the class II glucagon secretin family.

GLP-2 has been reported to induce significant growth of the small intestinal mucosal epithelium via the stimulation of stem cell proliferation in the crypts, and by inhibition of apoptosis in the villi (Drucker et al., 1996, Proc. Natl. Acad. Sci. USA 93: 7911-7916). GLP-2 also has growth effects on the colon. Furthermore, GLP-2 inhibits gastric emptying and gastric acid secretion (Wojdemann et al., 1999, J. Clin. Endocrinol. Metab. 84: 2513-2517), enhances intestinal barrier function (Benjamin et al., 2000, Gut 47: 112-119), stimulates intestinal hexose transport via the upregulation of glucose transporters (Cheeseman, 1997, Am. J. Physiol. R1965-71), and increases intestinal blood flow (Guan et al., 2003, Gastroenterology, 125: 136-147). For a review of GLP-2 and its properties, see Burrin et al., 2001, The Journal of Nutrition, 131(3), March 2001, 709-712.

It has been recognised in the art that glucagon-like peptide-2 receptor analogues have therapeutic potential for the treatment of intestinal diseases. However, the native hGLP-2, a 33 amino acid gastrointestinal peptide, is not a useful in a clinical setting due to its very short half-life in humans of around 7 minutes for full length GLP-2 [1-33] and 27 minutes for truncated GLP-2 [3-33]. In large part, the short half-life is due to degradation by the enzyme dipeptidylpeptidase IV (DPP-IV). Accordingly, there have been attempts in the art to develop GLP-2 receptor agonists with better pharmacokinetic characteristics, in particular to improve the half-life of GLP-2 molecules. By way of example, GLP-2 analogues with substitutions have been suggested such as e.g. GLP-2 analogues containing Gly substitution at position 2 ([hGly2] GLP-2, teduglutide) which increases the half-life from seven minutes (native GLP-2) to about two hours. Teduglutide is approved for treatment of short bowel syndrome under the names Gattex (in the US) and Revestive (in Europe).

WO 2006/117565 (Zealand Pharma A/S) describes GLP-2 analogues which comprise one of more substitutions as compared to [hGly2]GLP-2 and which improved biological activity in vivo and/or improved chemical stability, e.g. as assessed in in vitro stability assays.

Among the molecules disclosed in WO 2006/117565 is ZP1848 (glepaglutide) which has been designed to be stable in liquid formulations. Dosage regimes for GLP-2 analogues including ZP1848 and its metabolites are described in WO 2018/229252, which also shows that these compounds are effective to increase longitudinal growth of the intestines.

Use of GLP-2 analogues, including ZP1848, to treat conditions associated with bile acid synthesis, liver bile acid content, or intestinal bile acid content is described in WO 2020/020904.

Ready-to use formulations of ZP1848 are described in WO 2020/065064.

### SUMMARY OF THE INVENTION

Clearance of teduglutide is impaired in subjects with renal dysfunction, and in particular in patients with moderate or severe renal impairment, or end stage renal disease (ESRD). Consequently, it is recommended that the normal dose of teduglutide is reduced by 50% for patients with moderate renal impairment, severe renal impairment, or ESRD. See, for example, Nave, R et al., Eur. J. Clin. Pharmacol. 2013, 69(5): 1149-1155, Summary of Product Characteristics of Revestive® (European Medicines Agency), European Medicines Agency (2012) European Public Assessment Report of Revestive®.

Since ZP1848 is also a GLP-2 analogue, similar considerations might reasonably be expected to apply.

Surprisingly, though, it has been found that ZP1848 is cleared normally in patients with renal impairment. As a result, there is no need to adjust the dosage of ZP1848 depending on a patient's renal function. This provides a number of benefits. Firstly, it means that patients with impaired renal function are able to receive a full therapeutically effective dose without undesirable side effects, e.g. caused by over-exposure to the active agent. In addition, it obviates the need to test a patient's renal function before prescribing ZP1848, resulting in more efficient and economical treatment. Furthermore, safety should be improved in the event that a patient already receiving ZP1848 develops a form of renal impairment, since there should be no need to adjust their dose to take account of the renal event.

Thus, in a first aspect, the invention provides ZP1848 or a pharmaceutically acceptable salt thereof for use in prophylaxis or treatment of a condition responsive thereto in a subject with at least moderate renal impairment, wherein no dose adjustment of ZP1848 or said salt is required.

The invention further provides a method of prophylaxis or treatment of a condition responsive thereto in a subject with at least moderate renal impairment, comprising administering ZP1848 or a pharmaceutically acceptable salt thereof to said subject, wherein no dose adjustment of ZP1848 or said salt is required.

The invention further provides the use of ZP1848 or a pharmaceutically acceptable salt thereof in the preparation of a medicament for prophylaxis or treatment of a condition responsive thereto in a subject with at least moderate renal impairment, wherein no dose adjustment of ZP1848 or said salt is required.

The term “at least moderate” renal impairment is used in this specification to include subjects having moderate renal impairment, severe renal impairment, or ESRD.

Thus, the subject would typically require an adjusted dose of teduglutide, if teduglutide were to be prescribed for treatment of the same condition. The subject may or may not previously have been treated with teduglutide, e.g. at an adjusted dose.

In a further aspect, the invention provides ZP1848 or a pharmaceutically acceptable salt thereof for use in prophylaxis or treatment of a condition responsive thereto in a subject for whom an adjusted dose of teduglutide would be indicated as a result of renal impairment, wherein no dose adjustment of ZP1848 or said salt is required.

The invention further provides a method of prophylaxis or treatment of a condition responsive to ZP1848 in a subject for whom an adjusted dose of teduglutide would be indicated as a result of renal impairment, comprising administering ZP1848 or a pharmaceutically acceptable salt thereof to said subject, wherein no dose adjustment of ZP1848 or said salt is required.

The invention further provides the use of ZP1848 or a pharmaceutically acceptable salt thereof in the preparation of a medicament for prophylaxis or treatment of a condition responsive thereto in a subject for whom an adjusted dose of teduglutide would be indicated as a result of renal impairment, wherein no dose adjustment of ZP1848 or said salt is required.

Thus, the subject may have at least moderate renal impairment, e.g. moderate renal impairment, severe renal impairment, or ESRD.

The subject would typically require an adjusted dose of teduglutide, if teduglutide were to be prescribed for treatment of the same condition. The subject may or may not previously have been treated with teduglutide, e.g. at an adjusted dose.

In a further aspect, the invention provides ZP1848 or a pharmaceutically acceptable salt thereof for use in prophylaxis or treatment of a condition responsive thereto in a subject who has received an adjusted dose of teduglutide due to renal impairment, wherein no dose adjustment of ZP1848 or said salt is required.

The invention further provides a method of prophylaxis or treatment of a condition responsive to ZP1848 in a subject who has received an adjusted dose of teduglutide due to renal impairment, comprising administering ZP1848 or a pharmaceutically acceptable salt thereof to said subject, wherein no dose adjustment of ZP1848 or said salt is required.

The invention further provides the use of ZP1848 or a pharmaceutically acceptable salt thereof in the preparation of a medicament for prophylaxis or treatment of a condition responsive thereto in a subject who has received an adjusted dose of teduglutide due to renal impairment, wherein no dose adjustment of ZP1848 or said salt is required.

Thus the subject will previously have received teduglutide, at an adjusted dose, for treatment of the same condition. Typically, the subject has at least moderate renal impairment, e.g. moderate renal impairment, severe renal impairment, or ESRD.

The condition to be treated may be any condition therapeutically responsive to treatment with a GLP-2 analogue, e.g. where treatment results in amelioration of one or more symptoms, amelioration of underlying pathology, delay of onset, and/or inhibition of progression. Thus prophylaxis may be considered treatment or therapy.

Such conditions include stomach and bowel-related disorders such as ulcers, digestion disorders, malabsorption syndromes, short-gut syndrome, inflammatory bowel disease, celiac sprue (for example arising from gluten induced enteropathy or celiac disease), tropical sprue, hypogammaglobulinemic sprue, enteritis, regional enteritis (Crohn's disease), ulcerative colitis, small intestine damage or short bowel syndrome (SBS).

Use for the treatment of short bowel syndrome (SBS) may be of particular interest, especially in a subject receiving parenteral support (PS).

Further conditions include stomach and bowel-related disorders such radiation enteritis, infectious or post-infectious enteritis, or small intestinal damage due to toxic or other chemotherapeutic agents. In this case, treatment with the GLP-2 analogue may optionally be combined with one or more anti-cancer therapies, and may therefore comprise administering one or more chemotherapeutic agent(s) to the subject or treating the subject with radiation therapy.

Thus the condition may be a side effect of chemotherapy or radiation treatment in a human subject.

The terms “subject” and “patient” are used interchangeably in this specification. It will be understood that the subject (or patient) is a mammal, and typically a human.

ZP1848 is also effective in increasing intestinal mass and longitudinal intestinal growth, particularly in the small intestine.

Thus, in a further aspect, the invention provides ZP1848 or a pharmaceutically acceptable salt thereof for use in increasing intestinal mass and/or promoting or increasing longitudinal intestinal growth in a subject with at least moderate renal impairment, wherein no dose adjustment of ZP1848 or said salt is required.

The invention further provides a method of increasing intestinal mass and/or promoting or increasing longitudinal intestinal growth in a subject with at least moderate renal impairment, comprising administering ZP1848 or a pharmaceutically acceptable salt thereof to said subject, wherein no dose adjustment of ZP1848 or said salt is required.

The invention further provides the use of ZP1848 or a pharmaceutically acceptable salt thereof in the preparation of a medicament for increasing intestinal mass and/or promoting or increasing longitudinal intestinal growth in a subject with at least moderate renal impairment, wherein no dose adjustment of ZP1848 or said salt is required.

Thus, the subject would typically require an adjusted dose of teduglutide, if teduglutide were to be used for the same purpose. The subject may or may not previously have been treated with teduglutide, e.g. at an adjusted dose.

In a further aspect, the invention provides ZP1848 or a pharmaceutically acceptable salt thereof for use in increasing intestinal mass and/or promoting or increasing longitudinal intestinal growth in a subject for whom an adjusted dose of teduglutide would be indicated as a result of renal impairment, wherein no dose adjustment of ZP1848 or said salt is required.

The invention further provides a method of increasing intestinal mass and/or promoting or increasing longitudinal intestinal growth in a subject for whom an adjusted dose of teduglutide would be indicated as a result of renal impairment, comprising administering ZP1848 or a pharmaceutically acceptable salt thereof to said subject, wherein no dose adjustment of ZP1848 or said salt is required.

The invention further provides the use of ZP1848 or a pharmaceutically acceptable salt thereof in the preparation of a medicament for increasing intestinal mass and/or promoting or increasing longitudinal intestinal growth in a subject for whom an adjusted dose of teduglutide would be indicated as a result of renal impairment, wherein no dose adjustment of ZP1848 or said salt is required.

Thus, the subject may have at least moderate renal impairment, e.g. moderate renal impairment, severe renal impairment, or ESRD.

The subject would typically require an adjusted dose of teduglutide, if teduglutide were to be prescribed for the same purpose. The subject may or may not previously have been treated with teduglutide, e.g. at an adjusted dose.

In a further aspect, the invention provides ZP1848 or a pharmaceutically acceptable salt thereof for use in increasing intestinal mass and/or promoting or increasing longitudinal intestinal growth in a subject who has received an adjusted dose of teduglutide due to renal impairment, wherein no dose adjustment of ZP1848 or said salt is required.

The invention further provides a method of increasing intestinal mass and/or promoting or increasing longitudinal intestinal growth in a subject who has received an adjusted dose of teduglutide due to renal impairment, comprising administering ZP1848 or a pharmaceutically acceptable salt thereof to said subject, wherein no dose adjustment of ZP1848 or said salt is required.

The invention further provides the use of ZP1848 or a pharmaceutically acceptable salt thereof in the preparation of a medicament for increasing intestinal mass and/or promoting or increasing longitudinal intestinal growth in a subject who has received an adjusted dose of teduglutide due to renal impairment, wherein no dose adjustment of ZP1848 or said salt is required.

Thus the subject will previously have received teduglutide, at an adjusted dose, e.g. for the same purpose. Typically, the subject has at least moderate renal impairment, e.g. moderate renal impairment, severe renal impairment, or ESRD.

In such aspects, the subject may be affected by a condition in which increasing intestinal mass and/or promoting or increasing longitudinal intestinal growth is desirable, e.g. therapeutic. This may include any of the conditions set out elsewhere in this specification, but especially subjects affected by short bowel syndrome (SBS), e.g. those receiving parenteral support.

In a further aspect, the invention provides a pharmaceutical kit comprising ZP1848 or a pharmaceutically acceptable salt thereof and information that no dose adjustment of ZP1848 is required for subjects having at least moderate renal impairment.

The ZP1848 or pharmaceutically acceptable salt will typically be provided as a pharmaceutical composition, comprising ZP1848 or said salt in combination with a pharmaceutically acceptable carrier or excipient.

The kit may comprise one or more individual measured doses of ZP1848 or said salt, wherein each individual measured dose is an unadjusted dose as described elsewhere in this specification.

The individual doses may be for administration via a dosing regime as described elsewhere in this specification.

The kit may further comprise one or more chemotherapeutic agents, which may be provided in a separate pharmaceutical composition to ZP1848 or salt thereof.

It will be understood that subjects treated according to any aspect of the invention will typically receive ZP1848 or a pharmaceutically acceptable salt thereof instead of teduglutide (and any other GLP-2 receptor agonist), i.e. ZP1848 or the relevant salt will typically be the sole GLP-2 receptor agonist used for treatment of the subject. Similarly the compositions and kits defined herein typically contain ZP1848 or the relevant salt as the sole GLP-2 receptor agonist.

In the context of the present invention, an “adjusted” dose is a dose which has been reduced to take account of renal impairment. Thus, the dose of teduglutide is reduced by 50% when administered to subjects with at least moderate renal impairment to account for impaired clearance of the drug from the system of such patients, and to avoid over-exposure of the patient to the drug.

For ZP1848 and salts thereof, there is no need to adjust the dose to account for renal impairment in the patient. Thus, the dose for such subjects can be the same as that which would be provided to an otherwise equivalent subject with normal renal function. This may be referred to as the “normal”, “standard”, or “unadjusted” dose. It will be understood that a small amount of variation may nevertheless be permitted, e.g. +/−10%, without being considered an “adjusted” dose. Typically an “adjusted” dose would vary by more than 30%, e.g. more than 40%, e.g. about 50%.

It will, of course, be understood that the “normal” dose may be determined depending on the particular subject, e.g. depending on their age, sex, weight, disease status, etc. and/or on the intended dosage regime. Again, such variations should not be considered to result in an “adjusted” dose within the meaning of the invention. Rather, “adjusted” should be construed to mean “adjusted for considerations of renal function” or similar, unless the context demands otherwise.

Administration may be according to any suitable dosing regime.

For example, administration may be once daily.

However, as described in WO 2018/229252, ZP1848 has an unexpectedly long half-life which may enable alternative regimes such as once or twice weekly administration, especially when delivered by subcutaneous injection. Without wishing to be bound by theory, it is believed that the half life of ZP1848 may be due to the combination of the formation of a subcutaneous depot and the formation of metabolites which are slowly released from the subcutaneous depot and which are also agonistic on the GLP-2 receptor. The subcutaneous depot may be formed on administration through a reaction between the lysine tail of ZP1848 and hyaluronic acid in the subcutaneous compartment.

Thus the dosing regime may comprise a plurality or course of doses separated in time by 2 days, 2.5 days, 3 days, 3.5 days, 4 days, 5 days, 6 days, 7 days, 8 days, 9 days, 10 days, 11 days or 12 days. In a preferred embodiment, the doses are separated in time by 3 days, 3.5 days, 4 days, 5 days, 6 days, 7 days or 8 days. In a preferred embodiment, doses are separated in time by 3 days, 3.5 days, 4 days or 7 days. As will be appreciated in the art, the time between doses may be varied to some extent so that each and every doses is not separated by precisely the same time. This will often be directed under the discretion of the physician. Thus, doses may be separated in time by a clinically acceptable range of times, e.g. from about 2 days to about 10 days, or from about 3 or 4 days to about 7 or 8 days.

A typical “normal” or “unadjusted” dose may be in the range between 0.5 mg and 25 mg inclusive per subject per administration.

For example, it may be between 1 mg and 20 mg inclusive per subject per dose, e.g. between 1 mg and 10 mg inclusive per subject per dose, e.g. between 2 mg and 7 mg inclusive per subject per dose, e.g. between 5 mg and 7 mg inclusive per subject per dose, or between 2 mg and 5 mg inclusive per per subject per dose.

Alternatively, it may be between 5 mg and 15 mg inclusive per subject per dose, e.g. between 7 mg and 12 mg inclusive per subject per dose, e.g. between 9 mg and 11 mg inclusive per subject per dose.

In some embodiments, the dose of the GLP-2 analogues used in accordance with the present invention is about 10 mg per subject per dose (where “about” signifies +/−10%).

In some embodiments, the dose of the GLP-2 analogues used in accordance with the present invention is a fixed dose of 10 mg per subject.

Such doses may be appropriate for any dosage regime, but particularly a once or twice weekly regime.

In a course of treatment, the doses taken by the patient may either be the same or different in accordance with the instructions from the physician.

In some cases, it may be desirable to divide a total dose into a plurality (e.g. two or three) separate doses or administrations, for example for administration at spaced apart injection sites, for example spacing the injection sites at least 5 cm apart. Such spatially separate administrations will typically be provided at substantially the same time, e.g. on the same day, within one hour of each other, or even closer in time.

Embodiments of the present invention will now be described by way of example and not limitation However, various further aspects and embodiments of the present invention will be apparent to those skilled in the art in view of the present disclosure.

Unless context dictates otherwise, the descriptions and definitions of the features set out above are not limited to any particular aspect or embodiment of the invention and apply equally to all aspects and embodiments which are described.

### DETAILED DESCRIPTION OF THE INVENTION

Throughout the description and claims the conventional one-letter and three-letter codes for natural amino acids are used. All amino acid residues in the compounds described are typically of the L-configuration.

### Compounds

ZP1848 is a peptide having the formula:

### H-HGEGTFSSELATILDALAARDFIAWLIATKITDKKKKKK-NH₂

as described e.g. in WO 2006/117565. It will be understood that the N-terminal “H—” indicates a free N-terminal amine (NH₂—) group. The C-terminal “NH₂—” indicates a C-terminal amide group. The terms ZP1848 and glepaglutide may be used interchangeably.

The invention encompasses the use of pharmaceutically acceptable salts of ZP1848, as described in more detail below. Any suitable salt may be used, although acetate may be preferred.

When ZP1848 is injected into the subcutaneous (SC) compartment, two functionally active metabolites are formed, ZP2469 and ZP2711, both C-terminal truncated analogs of ZP1848. The overall PK profile of ZP1848 therefore comprises the effect of ZP1848 and its two main metabolites.

ZP2469 is a peptide having the formula:

### H-HGEGTFSSELATILDALAARDFIAWLIATKITDK-OH

ZP2711 is a peptide having the formula:

### H-HGEGTFSSELATILDALAARDFIAWLIATKITDKK-OH

where the N-terminal “H—” is as described above, and the C-terminal “—OH” indicates a free C-terminal carboxylic acid group.

Teduglutide is a peptide having the formula:

### H-HGDGSFSDEMNTILDNLAARDFINWLIQTKITD-OH

where the N-terminal “H—” and C-terminal “—OH” are as described above.

### Renal Function and Determination Thereof

Renal function is typically defined by reference to the glomerular filtration rate (GFR) or estimated glomerular filtration rate (eGFR).

GFR (units ml/min) may be determined using any appropriate filtration marker, such as inulin, ⁵¹Cr-EDTA, ^{99m}Tc-DTPA, iothalamate or iohexol. The skilled person will be well aware of suitable methods, e.g. based on monitoring excretion of the relevant marker in urine over a given time period such as 24 hours.

eGFR may be calculated from standardized serum creatinine (SCr) values. For example, it may be calculated according to the Modification of Diet in Renal Disease (MDRD) equation (Levey A S et al., Clin Chem. April 2007;53(4):766-772), which provides a value normalised to a body surface area of 1.73 m², in units of ml/min/1.73 m²:

eGFR=175×standardized SCr⁻1.154×age^{-0.203}×1.212 [if black]×0.742 [if female] [SCr in mg/dL]

or

eGFR=30849×standardized SCr⁻154×age^{-0.203}×1.212 [if black]×0.742 [if female] [SCr in μmol/L]

Alternatively, renal function may be defined by creatinine clearance (Ccr) using the Cockcroft-Gault equation (Cockcroft D W and Gault M H, Nephron 16: 31-41 (1976)):

Ccr=[(140−age)(wt kg)]/[72×SCr(mg/100 ml)]

for adult males; 15% less in adult females.

Alternative formulae may be used to calculate eGFR for children and adolescents (age 1-18), such as the creatinine-based “bedside Schwartz” equation (Schwartz G J and Work D F, J Am Soc Nephrol. 2009; November; 4(11): 1832-643; Schwartz G J et al., J Am Soc Nephrol. 2009; 20: 629-637):

eGFR=0.413×(height/SCr) [height expressed in cm; SCr mg/100 ml]

For adults, it may be preferred to calculate eGFR using the MDRD equation.

For the purposes of calculating eGFR, standardised serum creatinine levels may be determined by isotope dilution gas chromatography/mass spectrometry (ID-GC/MS), e.g. as described by Stoeckl and Reinauer, Clin. Chem. 1993;39:993-1000, which represents the “gold standard” for creatinine measurement.

It will be appreciated that other methods and commercial kits are available for measuring serum creatinine, including enzymatic methods, or colorimetric methods such as the Jaffé reaction in which creatinine forms a coloured product after reaction with alkaline picrate. Such methods typically employ various compensation or correction factors to more accurately reflect the gold standard results, e.g. by minimising interference from bilirubin and pseudo-creatinine chromogens such as proteins and ketones. Examples include the Cobas® CREJ2 (Creatinine Jaffe second generation) kit (Roche).

The current classifications of renal function for dedicated renal impairment studies can be found in the “Guidance for Industry Pharmacokinetics in Patients with Impaired Renal Function—Study Design, Data Analysis, and Impact on Dosing, U.S. Department of Health and Human Services, Food and Drug Administration, Center for Drug Evaluation and Research (CDER), September 2020” or the corresponding European “Guideline on the evaluation of the pharmacokinetics of medicinal products in patients with decreased renal function, European Medicines Agency, 2015”.

Under current clinical guidelines, normal renal function may be defined as GFR (or Ccr)≥90 mL/min or eGFR≥90 mL/min/1.73 m².

Mild renal impairment may be defined as GFR (or Ccr) 60 to <90 mL/min, or eGFR 60 to <90 mL/min/1.73 m²

Moderate renal impairment may be defined as GFR (or Ccr) 30 to <60 mL/min or eGFR 30 to <60 mL/min/1.73 m²

Severe renal impairment may be defined as GFR (or Ccr) 15 to <30 mL/min or eGFR 15 to <30 mL/min/1.73 m²

End stage renal disease (ESRD) is typically characterised by GFR (or Ccr) <15 mL/min or eGFR<15 mL/min/1.73 m².

Previous guidelines placed the threshold for moderate renal impairment at <50 mL/min rather than <60 mL/min, and original guidance for teduglutide consequently indicated a 50% dose reduction for patients having GFR<50 mL/min. Thus it will be understood that no dose adjustment is required for subjects having GFR (or Ccr)<50mL/min or eGFR<50 mL/min/1.73 m². Under certain circumstances it may be appropriate to regard this value as the threshold for moderate renal impairment. Thus, subjects having at least moderate renal impairment may be considered to be those having GFR (or Ccr)<50mL/min or eGFR<50 mL/min/1.73 m².

For the purposes of the invention it may be preferred to base a subject's classification on a measurement of eGFR, calculated for example by the MDRD equation, based on measurement of standardised SCr. SCr is preferably determined by ID-GC/MS, e.g. as described above.

### Pharmaceutical Compositions and Administration

The active agents described may be formulated as pharmaceutical compositions prepared for storage or administration, and which comprise a therapeutically effective amount of the active agent in a pharmaceutically acceptable carrier.

The therapeutically effective amount of the relevant active agent will depend on the route of administration, the type of mammal being treated (typically human), and the physical characteristics of the specific mammal under consideration. These factors and their relationship to determining this amount are well known to skilled practitioners in the medical arts. This amount and the method of administration can be tailored to achieve optimal efficacy so as to deliver the peptide to the intestine, but will depend on such factors as weight, diet, concurrent medication and other factors, well known those skilled in the medical arts.

The active agent is typically present in an amount effective for prophylaxis or treatment of the relevant condition, e.g. to treat or prevent stomach and bowel-related disorders, to increase intestinal mass and/or to promote or increase longitudinal intestinal growth of the intestines in a subject.

Examples of pharmaceutically acceptable salts are described in “Remington's Pharmaceutical Sciences”,17^{th} edition. Ed. Alfonso R. Gennaro (Ed.), Mark Publishing Company, Easton, PA, U.S.A., 1985 and more recent editions, and in the Encyclopaedia of Pharmaceutical Technology.

Suitable salts include acid addition salts and basic salts. Examples of acid addition salts include hydrochloride salts, citrate salts, chloride salts and acetate salts. Preferably, the salt is acetate. In general, it is preferred that the salt is not a chloride salt. Examples of basic salts include salts where the cation is selected from alkali metals, such as sodium and potassium, alkaline earth metals, such as calcium, and ammonium ions ⁺N (R³)₃(R⁴) where R³ and R⁴ independently designates optionally substituted C₁₋₆-alkyl, optionally substituted C₂₋₆-alkenyl, optionally substituted aryl, or optionally substituted heteroaryl.

Acetate salts may be particularly preferred. In the present context, the term “ZP1848-acetate” refers to the ZP1848 molecule is in the form of an acetate salt. The acetate salts of ZP1848 may be represented by the formula (ZP1848), x(CH₃COOH) where x is 1.0 to 8.0, i.e. where x is 1.0, 2.0, 3.0, 4.0, 5.0, 6.0, 7.0 or 8.0. In any composition, there may be molecules with different number of acetate molecules so that x is not necessarily a whole integer. In some cases, x is from 4.0 to 8.0, x is from 6.0 to 8.0, or x is from 4.0 to 6.5. In some cases is from x is from 4.0 to 6.0, x is from 2.0 to 7.0, x is from 3.0 to 6.0, x is from 4.0 to 6.0 or x is 4.0 to 8.0.

As is apparent to one skilled in the medical art, a “therapeutically effective amount” of the peptides or pharmaceutical compositions of the present invention may vary depending upon the age, weight and mammalian species treated, the particular compounds employed, the particular mode of administration and the desired effects and the therapeutic indication. Because these factors and their relationship to determining this amount are well known in the medical arts, the determination of therapeutically effective dosage levels, the amount necessary to achieve the desired result (e.g. of preventing and/or treating the intestine and stomach related diseases described herein, as well as other medical indications disclosed herein, or increasing intestinal mass and/or inducing or increasing longitudinal intestinal growth of the intestines in a subject) will be within the ambit of the skilled person.

As used herein, “a therapeutically effective amount” is one which reduces symptoms of a given condition or pathology, and preferably which normalizes physiological responses in an individual with the condition or pathology. Reduction of symptoms or normalization of physiological responses can be determined using methods routine in the art and may vary with a given condition or pathology. In one aspect, a therapeutically effective amount is an amount which restores a measurable physiological parameter to substantially the same value (preferably to within +30%, more preferably to within +20%, and still more preferably, to within 10% of the value) of the parameter in an individual without the condition or pathology.

In one embodiment of the invention administration of the compounds or pharmaceutical composition of the present invention is commenced at lower dosage levels, with dosage levels being increased until the desired effect of preventing/treating the relevant medical indication, such as intestine and stomach related diseases or increased longitudinal growth of the intestines, is achieved. This would define a therapeutically effective amount. Guidance on appropriate individual doses is provided elsewhere in this specification. However, the skilled person will be able to adjust these doses in the event that an alternative dosing regime is selected.

For therapeutic use, the active agent is formulated with a carrier that is pharmaceutically acceptable and is appropriate for delivering the peptide by the chosen route of administration. For the purpose of the present invention, peripheral parenteral routes include intravenous, intramuscular, subcutaneous, and intraperitoneal routes of administration. In one embodiment, the route of administration is the subcutaneous route or subcutaneous administration.

When administration is to be parenteral, such as intravenous, subcutaneous or intramuscular injectable pharmaceutical compositions can be prepared in conventional forms, either as aqueous solutions or suspensions; lyophilized, solid forms suitable for reconstitution immediately before use or suspension in liquid prior to injection, or as emulsions.

Diluents for reconstitution of the lyophilized product may be a suitable buffer, e.g. selected from a histidine buffer, mesylate buffer, acetate buffer, glycine buffer, lysine buffer, TRIS buffer, Bis-Tris buffer and MOPS buffer, water, saline, dextrose, mannitol, lactose, trehalose, sucrose, lecithin, albumin, sodium glutamate, cysteine hydrochloride; or water for injection with addition of detergents, such as Tween 20, Tween 80, poloxamers e.g. pluronic F-68 or pluronic F-127, polyethylene glycol, and or with addition of preservatives such as para-, meta-, and ortho-cresol, methyl- and propylparaben, phenol, benzyl alcohol, sodium benzoate, benzoic acid, benzyl-benzoate, sorbic acid, propanoic acid, esters of p-hydroxybenzoic acid, and or with addition of an organic modifier such as ethanol, acetic acid, citric acid, lactic acid or salts thereof.

In addition, if desired, the injectable pharmaceutical compositions may contain minor amounts of non-toxic auxiliary substances, such as wetting agents, or pH buffering agents. Absorption enhancing preparations (e.g., liposomes, detergents and organic acids) may be utilized.

In one embodiment of the invention, the compounds are formulated for administration by infusion, e.g., when used as liquid nutritional supplements for patients on total parenteral nutrition therapy (for example neonatals, or patients suffering from cachexia or anorexia), or by injection, for example subcutaneously, intraperitoneal or intravenously, and are accordingly utilized as aqueous solutions in sterile and pyrogen-free form and optionally buffered to physiologically tolerable pH, e.g., a slightly acidic or physiological pH. Formulation for intramuscular administration may be based on solutions or suspensions in plant oil, e.g. canola oil, corn oil or soy bean oil. These oil based formulations may be stabilized by antioxidants e.g. BHA (butylated hydroxianisole) and BHT (butylated hydroxytoluene).

Thus, the present peptide compounds may be administered in a vehicle, such as distilled water or in saline, phosphate buffered saline, 5% dextrose solutions or oils. The solubility of the active agent may be enhanced, if desired, by incorporating a solubility enhancer, such as detergents and emulsifiers.

The aqueous carrier or vehicle can be supplemented for use as injectables with an amount of gelatin that serves to depot the active agent at or near the site of injection, for its slow release to the desired site of action. Alternative gelling agents, such as hyaluronic acid, may also be useful as depot agents.

Subcutaneous administration may be particularly preferred, e.g. by injection.

The active agents may also be formulated as a slow release implantation device for extended and sustained administration. Such sustained release formulations may be in the form of a patch positioned externally on the body. Examples of sustained release formulations include composites of biocompatible polymers, such as poly(lactic acid), poly(lactic-co-glycolic acid), methylcellulose, hyaluronic acid, sialic acid, silicate, collagen, liposomes and the like. Sustained release formulations may be of particular interest when it is desirable to provide a high local concentration of active agent.

The therapeutic dosing and regimen most appropriate for patient treatment will of course vary with the disease or condition to be treated, and according to the patient parameters. Without wishing to be bound by any particular theory, it is expected that doses, between 0.1 and 25 mg per patient, and shorter or longer duration or frequency of treatment may produce therapeutically useful results, such as a statistically significant increase particularly in small bowel mass. In some instances, the therapeutic regimen may include the administration of maintenance doses appropriate for preventing tissue regression that occurs following cessation of initial treatment. The dosage sizes and dosing regimen most appropriate for human use may be guided by the results obtained by the present invention, and may be confirmed in further clinical trials.

A human dose of ZP1848 may be used in a dose of between about 0.01 mg/kg and 100 mg/kg body weight, such as between about 0.01 mg/kg and 10 mg/kg body weight, for example between 10-100 μg/kg body weight. In further embodiments, a human dose (total dose) of ZP1848 may be from about such as between and including 0.1 mg and 25 mg per 30 patient between and including 0.5 mg and 20 mg per patient, such as between and including 1 mg and 15 mg per patient, such as between and including 1 mg and 10 mg per patient once or twice weekly or as a plurality of doses as defined herein separated in time by 2, 3, 4, 5, 6, 7, 8, 9, 10, 11, 12, 13 or 14 days. In some instances, a fixed dose of ZP1848 may be used in accordance with a dosing pattern disclosed herein, i.e. a dose which is the same regardless of the body weight of the patient, given once or twice weekly. By way of example, the fixed dose may be a dose of 5 mg, 6 mg, 7 mg, 8 mg, 9, mg, 10 mg, 11 mg, 12 mg, 13 mg, 14 mg or 15 mg. Conveniently a fixed dose of 10 mg may be used. The use of fixed dosing has the advantage of increasing compliance and reducing the risk of patient dosing errors, including risks of miscalculating a weight based dose to be administered.

In preferred embodiments, the formulation is a ready-to-use formulation as described in

WO 2020/065064. The term “ready-to-use” as used herein refers to a formulation that does not require constitution or dilution with a prescribed amount of diluent, e.g., water for injection or other suitable diluent, before use by the designated route of administration.

As described herein, the liquid formulations of the GLP-2 analogues of the present invention include a buffer, a non-ionic tonicity modifier and arginine q.s. to provide the pH of the final formulation. In accordance with normal pharmaceutical practice, the formulations of the present invention are sterile and/or free from reducing agent. In some cases, the liquid formulations of the present invention are aqueous, liquid formulations. In some cases, the liquid formulations of the present invention are non-aqueous, liquid formulations.

The term “buffer” as used herein denotes a pharmaceutically acceptable excipient which stabilizes the pH of a pharmaceutical formulation. Suitable buffers are well known in the art and can be found in the literature. The screening experiments in the examples show that the formulations of the present invention preferably include a buffer selected from a histidine buffer, mesylate buffer, acetate buffer, glycine buffer, lysine buffer, TRIS buffer, Bis-Tris buffer and MOPS buffer as these buffers provided stable formulations in which the GLP-2 analogues dissolved and did not become viscous, cloudy or precipitate the peptide drug. In preferred embodiments, the buffer is a histidine buffer, e.g. L-histidine. Generally, the buffer will be present at a concentration of about 5 mM to about 50 mM, more preferably at a concentration of about 5 mM to about 25 mM, and most preferably at a concentration of about 15 mM. Preferably the buffer is not a phosphate buffer, a citrate buffer, citrate/Tris buffer and/or succinate buffer.

The term “tonicity modifier” as used herein denotes pharmaceutically acceptable tonicity agents that are used to modulate the tonicity of the formulation. The formulations of the present invention are preferably isosmotic, that is they have an osmotic pressure that is substantially the same as human blood serum. The tonicity modifiers used in the formulations are preferably non-ionic tonicity modifiers and are preferably selected from the group consisting of mannitol, sucrose, glycerol, sorbitol and trehalose. A preferred non-ionic tonicity modified is mannitol, e.g. D-mannitol. The concentration of the tonicity modifier will be dependent on the concentration of other components of the formulation, especially where the formulation is intended to be isosmotic. Typically, the non-ionic tonicity modifier will be employed at a concentration of about 90 mM to about 360 mM, more preferably at a concentration of about 150 mM to about 250 mM, and most preferably at a concentration of about 230 mM.

Generally, the components and amounts of the liquid formulations are chosen to provide a formulation with a pH of about 6.6 to about 7.4, more preferably a pH of about 6.8 to about 7.2, and most preferably a pH of about 7.0. Arginine may be added quantum sufficit (q.s.) to adjust pH so that it is within a desired pH range. From the experiments shown in the examples, it is preferred that the pH adjustment is not done using hydrochloric acid or sodium hydroxide.

In one embodiment, the liquid formulations consist of ZP1848, e.g. an acetate salt thereof, at a concentration of about 2 mg/mL to about 30 mg/mL a buffer selected from the group consisting of a histidine buffer, mesylate buffer, acetate buffer, glycine buffer, lysine buffer, TRIS buffer, Bis-Tris buffer and MOPS buffer, the buffer being present at a concentration of about 5 mM to about 50 mM, a non-ionic tonicity modifier selected from the group consisting of mannitol, sucrose, glycerol, sorbitol and trehalose at a concentration of about 90 mM to about 360 mM, arginine q.s. to provide a pH of about 6.6 to about 7.4.

In one embodiment, the liquid formulations consist of ZP1848, e.g. an acetate salt thereof, at a concentration of about 2 mg/mL to about 30 mg/mL, a buffer selected from the group consisting of a histidine buffer, mesylate buffer and acetate buffer, the buffer being present at a concentration of about 5 mM to about 50 mM, a non-ionic tonicity modifier selected from the group consisting of mannitol, sucrose, glycerol and sorbitol at a concentration of about 90 mM to about 360 mM, arginine q.s. to provide a pH of about 6.6 to about 7.4.

In a further embodiment, the liquid formulations comprise ZP1848, e.g. an acetate salt thereof, at a concentration of about 20 mg/mL, histidine buffer at a concentration of about 15 mM, mannitol at a concentration of about 230 mM, and arginine q.s. to provide a pH of about 7.0.

In a further embodiment, the liquid formulations comprise ZP1848, e.g. an acetate salt thereof, at a concentration of about 20 mg/mL, histidine buffer at a concentration of about 15 mM, mannitol at a concentration of about 230 mM and the pH is about 7.0.

In a further embodiment, the liquid formulations comprise ZP1848-acetate or H-HGEGTFSSELATILDALAARDFIAWLIATKITDKKKKKK-NH₂ acetate (SEQ ID NO: 1) at a concentration of about 20 mg/mL, histidine buffer at a concentration of about 15 mM, mannitol at a concentration of about 230 mM, and arginine q.s. to provide a pH of about 7.0.

In a further embodiment, the liquid formulations comprise ZP1848-acetate or H-HGEGTFSSELATILDALAARDFIAWLIATKITDKKKKKK-NH₂ acetate (SEQ ID NO: 1) at a concentration of about 20 mg/mL, histidine buffer at a concentration of about 15 mM, mannitol at a concentration of about 230 mM and the pH is about 7.0.

In a further embodiment, the liquid formulations comprise an acetate salt of a glucagon-like peptide 2 (GLP-2) analogue having the formula:

(H-HGEGTFSSELATILDALAARDFIAWLIATKITDKKKKKK-NH₂), x(CH₃COOH) where x is 1.0 to 8.0., at a concentration of about 20 mg/mL, histidine buffer at a concentration of about 15 mM, mannitol at a concentration of about 230 mM and the pH is about 7.0.

In a further embodiment, the liquid formulations comprise an acetate salt of a glucagon-like peptide 2 (GLP-2) analogue having the formula:

(H-HGEGTFSSELATILDALAARDFIAWLIATKITDKKKKKK-NH2), x(CH₃COOH) where x is 1.0 to 8.0., at a concentration of about 20 mg/mL, histidine buffer at a concentration of about 15 mM, mannitol at a concentration of about 230 mM and the pH is about 7.0, in a once or twice daily dosing regimen.

In a further embodiment, the liquid formulations comprise an acetate salt of a glucagon-like peptide 2 (GLP-2) analogue having the formula:

(H-HGEGTFSSELATILDALAARDFIAWLIATKITDKKKKKK-NH2), x(CH₃COOH) where x is 1.0 to 8.0., at a concentration of about 20 mg/mL, histidine buffer at a concentration of about 15 mM, mannitol at a concentration of about 230 mM and the pH is about 7.0, in a once or twice weekly dosing regimen.

In some cases, the liquid formulations of the present invention further comprise a preservative. In some cases, the preservative is one selected from the group consisting of benzalkonium chloride, chloro butanol, methyl paraben and potassium sorbate. Generally, the preservative is present in a concentration of about 0.1% to about 1% of the final formulation volume.

### Medical Conditions

The peptides of the present invention are useful as a pharmaceutical agent for preventing or treating an individual suffering from gastro-intestinal disorders, including the upper gastrointestinal tract of the oesophagus by administering an effective amount of a ZP1848, or a salt thereof as described herein. The stomach and intestinal-related disorders include ulcers of any aetiology (e.g., peptid ulcers, drug-induced ulcers, ulcers related to infections or other pathogens), digestion disorders, malabsorption syndromes, short-bowel syndrome, inflammatory bowel disease, celiac sprue (for example arising from gluten induced enteropathy or celiac disease), tropical sprue, hypogammaglobulinemic sprue, enteritis, ulcerative colitis, small intestine damage, and chemotherapy induced diarrhea/mucositis (CID).

As mentioned above in general, individuals who would benefit from increased small intestinal mass and consequent and/or maintenance of normal small intestine mucosal structure and function are candidates for treatment with ZP1848 or a salt thereof. Particular conditions that may be treated with ZP1848 include the various forms of sprue including celiac sprue which results from a toxic reaction to alpha-gliadin from heat and may be a result of gluten-induced enteropathy or celiac disease, and is marked by a significant loss of villae of the small bowel; tropical sprue which results from infection and is marked by partial flattening of the villae; hypogammaglobulinemic sprue which is observed commonly in patients with common variable immunodeficiency or hypogammaglobulinemia and is marked by significant decrease in villus height. The therapeutic efficacy of the ZP1848 or a salt thereof treatment may be monitored by enteric biopsy to examine the villus morphology, by biochemical assessment of nutrient absorption, by patient weight gain, or by amelioration of the symptoms associated with these conditions.

Another particular condition which may be treated with ZP1848 or a salt thereof, or for which ZP1848 or a salt thereof may be useful therapeutically and/or prophylactically is short bowl syndrome (SBS), also known as short gut syndrome or simply short gut, which results from surgical resection, congenital defect or disease-associated loss of absorption in the bowel in which patients are subsequently unable to maintain fluid, electrolyte, and nutrient balances on a conventional diet. Despite an adaptation that occurs generally in the two years after resection, SBS patients have reduced dietary uptake and fluid loss.

The class of human patients with SBS includes patients having SBS-intestinal failure (SBS-IF) and patients who are on the border between having SBS-intestinal insufficiency (SBS-II) and SBS-intestinal failure (SBS-IF). In some cases, patients having SBS-intestinal failure (SBS-IF) are also called SBS-PS when they are dependent on parenteral support, and the patients having SBS-intestinal insufficiency (SBS-II) are also called SBS non-PS if they are not depending on parenteral support.

The spectrum of patient types with SBS is reviewed in Jeppensen, Journal of Parenteral and Enteral Nutrition, 38(1), 8S-13S, May 2014, doi: 10.1177/0148607114520994. A further division of SBS patient types can be made along the lines described in Schwartz et al., Clinical and Translational Gastroenterology (2016) 7, e142; doi:10.1038/ctg.2015.69. This separates SBS patients into early responders and late/slow responders. It is presently believed that the early responders are the ones who exhibit an early effect on treatment with a GLP-2 analogue such as ZP1848 caused by, among other effects, an increase in the width/diameter of the small intestine, while the late or slow responders are the patients which mostly or first benefit to the treatment with a GLP-2 analogue caused by an increase in the length of the small intestine. The determination of whether a subject is an early or a late responder may be used to determine the duration of the treatment regime with the GLP-2 analogue, the timing of any clinical decision to reduce parenteral support and the interval between testing to determine whether a reduction in parenteral support is possible. Accordingly, in one embodiment, the patient is a late or slow responder. The length of the small intestines may for example be measured by CT scan (computed tomography scan), MRI (magnetic resonance imaging), histology, laparoscopic or other measurements or techniques known in the art.

In the present context, the term “parenteral support” or “PS” includes the provision of nutrients and/or fluids to the subject receiving GLP-2 therapy as a means of providing the subject with the nutrients and/or fluids that they require, but are unable to absorb fully due to their condition.

Other conditions that may be treated, or for which ZP1848 or a salt thereof may be useful prophylactically, include radiation enteritis, infectious or post-infectious enteritis, and small intestinal damage due to cancer-chemotherapeutic or toxic agents.

This may require administration of ZP1848 or a salt thereof prior to, concurrently with or following a course of chemotherapy or radiation therapy in order to reduce side effects of chemotherapy such as diarrhoea, abdominal cramping and vomiting, and reduce the consequent structural and functional damage of the intestinal epithelium resulting from the chemotherapy or radiation therapy. Preferably, administration is initiated 1, 2, 3, 4, 5, 6 or 7 days prior to the initiation of the chemotherapy or radiation cycle. Preferably, administration is initiated the day before or same day as start of treatment with chemotherapy or radiation cycle and once or twice weekly thereafter.

Intestinal damage and dysfunction is a well-known side effect of cancer-chemotherapy treatment. Chemotherapy administration is frequently associated with unwanted side effects related to the gastronintestinal system such as mucositis, diarrhoea, bacterial translocation, malabsorption, abdominal cramping, gastrointestinal bleeding and vomiting. These side effects are clinical consequences of the structural and functional damage of the intestinal epithelium and frequently make it necessary to decrease the dose and frequency of chemotherapy. Administration of ZP1848 or a salt thereof may enhance trophic effect in the intestinal crypts and rapidly provide new cells to replace the damaged intestinal epithelium following chemotherapy. The ultimate goal is to reduce the morbidity related to gastrointestinal damage of patients undergoing chemotherapy treatment while creating the most optimal chemotherapy regime for the treatment of cancer. Concomitant prophylactic or therapeutic treatment may be provided in accordance with the present invention to patients undergoing or about to undergo radiation therapy.

The stem cells of the small intestinal mucosa are particularly susceptible to the cytotoxic effects of chemotherapy due to their rapid rate of proliferation (Keefe et al., Gut 2000; 47: 632-7). Chemotherapy-induced damage to the small intestinal mucosa is clinically often referred to as gastrointestinal mucositis and is characterized by absorptive and barrier impairments of the small intestine. For example, it has been shown that, the broadly used chemotherapeutic agents, 5-FU, irinotecan and methothrexate increase apoptosis leading to villus atrophy and crypt hypoplasia in the small intestine of rodents (Keefe et al., Gut 47: 632-7, 2000; Gibson et al., J Gastroenterol Hepatol. September;18(9):1095-1100, 2003; Tamaki et al., J Int Med Res. 31(1):6-16, 2003). Chemotherapeutic agents have been shown to increase apoptosis in intestinal crypts at 24 hours after administration and subsequently to decrease villus area, crypt length, mitotic count per crypt, and enterocyte height three days after chemotherapy in humans (Keefe et al., Gut 2000; 47: 632-7). Thus, structural changes within the small intestine directly lead to intestinal dysfunction and in some cases diarrhea.

Gastrointestinal mucositis after cancer chemotherapy is an increasing problem that is essentially untreatable once established, although it gradually remits. Studies conducted with the commonly used cytostatic cancer drugs 5-FU and irinotecan have demonstrated that effective chemotherapy with these drugs predominantly affects structural integrity and function of the small intestine while the colon is less sensitive and mainly responds with increased mucus formation (Gibson et al., J Gastroenterol Hepatol. September;18(9):1095-1100, 2003; Tamaki et al., J Int Med Res. 31(1):6-16, 2003).

ZP1848 may be useful in the prevention and/or treatment of gastrointestinal injury and side effects of chemotherapeutic agents. This potentially important therapeutic application may apply to currently used chemotherapeutic agents such as but not limited to: 5-FU, Altretamine, Bleomycin, Busulfan, Capecitabine, Carboplatin, Carmustine, Chlorambucil, Cisplatin, Cladribine, Crisantaspase, Cyclophosphamide, Cytarabine, Dacarbazine, Dactinomycin, Daunorubicin, Docetaxel, Doxorubicin, Epirubicin, Etoposide, Fludarabine, Fluorouracil, Gemcitabine, Hydroxycarbamide, Idarubicin, Ifosfamide, Irinotecan, Liposomal doxorubicin, Leucovorin, Lomustine, Melphalan, Mercaptopurine, Mesna, Methotrexate, Mitomycin, Mitoxantrone, Oxaliplatin, Paclitaxel, Pemetrexed, Pentostatin, Procarbazine, Raltitrexed, Streptozocin, Tegafur-uracil, Temozolomide, Thiotepa, Tioguanine/Thioguanine, Topotecan, Treosulfan, Vinblastine, Vincristine, Vindesine, Vinorelbine, Bleomycin, Busulfan, Capecitabine, Carboplatin, Carmustine, Chlorambucil, Cisplatin, Cladribine, Crisantaspase, Cyclophosphamide, Cytarabine, Dacarbazine, Dactinomycin, Daunorubicin, Docetaxel, Doxorubicin, Epirubicin, Etoposide, Fludarabine, Fluorouracil, Gemcitabine, Hydroxycarbamide, Idarubicin, Ifosfamide, Irinotecan, Liposomal doxorubicin, Leucovorin, Lomustine, Melphalan, Mercaptopurine, Methotrexate, Mitomycin, Mitoxantrone, Oxaliplatin, Paclitaxel, Pemetrexed, Pentostatin, Procarbazine, Raltitrexed, Streptozocin, Tegafur-uracil, Temozolomide, Thiotepa, Tioguanine/Thioguanine, Topotecan, Treosulfan, Vinblastine, Vincristine, Vindesine, and Vinorelbine.

Further aspects of the invention relate to increasing the intestinal mass or longitudinal growth of the intestines in a patient, e.g. in a human patient, especially the small intestine. ZP1848 or a salt thereof is capable of increasing the longitudinal growth of the intestines relative to a control treatment, as shown in WO 2018/229252.

This capability is of particular value in patients with SBS as this will lead to increased absorptive capacity also after treatment is stopped. Such patient would be treated for at least 1 to 3 years, such as at least 1 to 4 years, such as 1 to 10 years, such as 1 to 20 years, such as 1 to 35 years with the objective of inducing longitudinal growth of the intestines.

As already described herein, SBS patients who are on the border between intestinal insufficiency (SBS-II) or SBS-PS patients and intestinal failure (SBS-IF) or SBS non-PS may therefore have particular value from having their intestines lengthened over a 1 to 3 year treatment course, whereafter their risk if intestinal failure is decreased, for example involving weekly or twice weekly dosing over the period of treatment. This involves less risk for central catheter needs and the risk of sepsis associated with its use.

The active agents may also be used for the treatment of malnutrition, for example resulting from cachexia and anorexia.

### EXAMPLES

The following examples are provided to illustrate preferred aspects of the invention and are not intended to limit the scope of the invention. The GLP-2 analogues administered according to the dosage regimes described herein can be made according to the methods such as solid phase peptide synthesis described in WO 2006/117565, the content of which is expressly incorporated by reference in its entirety.

### Example 1: Pharmacokinetic (PK) Profile of Glepaglutide and its Two Main Active Metabolites in Subjects With Varying Degrees of Renal Function

### Purpose

Glepaglutide (ZP1848, glepaglutide₁₋₃₉) is a potent long acting GLP-2 analogue currently in phase 3 for the treatment of short bowel syndrome (SBS). Glepaglutide is comprised of 39 L-amino acids, all of which are naturally occurring. Glepaglutide has 9 amino acid substitutions compared to native GLP-2 and a C-terminal tail consisting of 6 lysine residues, all which enables a stable long-lasting liquid formulation.

Following subcutaneous injection of glepaglutide, two functionally active metabolites i.e. ZP2469 (1848₁₋₃₄) and ZP2711 (ZP1848₁₋₃₅) are formed from cleavages within the C-terminus.

Patients with short bowel syndrome are at risk of suffering from renal impairment, where dose adjustment of glepaglutide could be warranted, hence a clinical phase 1 trial was conducted to investigate the pharmacokinetics of glepaglutide after a single dose in subjects with renal impairment compared to subjects with a normal renal function.

The clinical study protocol, its amendments/updates, the informed consent forms (ICFs) and their amendments were reviewed and approved by an Independent Ethics Committee (IEC) prior to the screening.

### Methods

The study was designed as a two-stage design, open-label, multi-center, non-randomized trial evaluating the PK of a single, subcutaneous dose of 10 mg glepaglutide in subjects with varying degrees of renal function. The renal function was calculated by the estimated glomerular filtration rate (eGFR) according to the Modification of Diet in Renal Disease (MDRD) equation.

Sixteen Caucasian subjects were enrolled, 4 with end stage renal disease (ESRD) not on dialysis (eGFR<15 mL/min), 4 with severe renal impairment (eGFR<30 mL/min) and 8 matching controls with normal renal function (eGFR>90 mL/min) (Table 1). Demographics, except eGFR, was similar across groups (Table 1).

Glepaglutide (ZP1848, 10 mg) was provided in a single-use vial containing 1 mL (an extractable volume of 0.5 mL) of a clear, colourless solution for subcutaneous injection, with a concentration of 20 mg/mL glepaglutide.

PK-blood samples were collected over a 14 day period following a single dose of 10 mg glepaglutide in the abdomen. The PK-samples were analyzed for glepaglutide, ZP2469 and ZP2711 using a GLP-validated LC/MS/MS assay.

The primary PK parameters were area under the curve between dose and last measurable concentration (AUCₜ), extrapolated to infinity (AUC_{inf}) and calculated from 0 to 168 hours (AUC₀₋₁₆₈) and the maximum plasma concentration (Cmax), which were computed for glepaglutide, ZP2469, ZP2711. A constructed analyte “glepaglutide-total” (glepaglutide+ZP2469+ZP2711) was used for the primary endpoint, and analysed using a non-compartmental approach.

### Results

There were no statistically significant differences in the primary PK parameters in subjects with severe renal impairment and ESRD, when compared with the healthy matched subjects. In particular, there was no clinically relevant difference between subjects with severe renal impairment/ESRD and normal renal function with respect to total exposure (AUC₀₋₁₆₈ₕ) and peak plasma concentrations (Cₘₐₓ) of glepaglutide following a single SC dose.

Geometric mean ratios for glepaglutideₜₒₜₐₗ were 0.96 [90% Cl:0.69-1.35] for AUC₀₋₁₆₈ and 0.90 [90% Cl: 0.62-1.31] for Cₘₐₓ. Hence, the exposure of glepaglutideₜₒₜₐₗ in subjects with renal impairment was 4% and 10% lower compared to healthy subjects for the two PK parameters, respectively, which is not considered clinically relevant.

The exposure of glepaglutideₜₒₜₐₗ was similar in subjects with renal impairment and healthy subjects with normal renal function. This suggests that renal function is not affecting the systemic exposure of glepaglutide, and therefore dose adjustment of glepaglutide in patients with renal impairment is not needed.

Geometric mean ratios for ZP2469 were 0.96 [90% Cl:0.667-1.38] for AUC₀₋₁₆₈ and 0.91 [90% Cl: 0.595-1.39] for Cₘₐₓ.

Geometric mean ratios for ZP2711 were 0.89 [90% Cl:0.616-1.29] for AUC₀₋₁₆₈ and 0.81 [90% Cl: 0.572-1.15] for Cₘₐₓ.

TABLE 1

| | Healthy | Severe/ESRD |
|---|---|---|
| Gender | 6 male, 2 female | 6 male, 2 female |
| Age Mean (years) | 46.4 | 46.5 |
| Weight Mean (kg) | 79.2 | 79.8 |
| BMI Mean (kg/m²) | 26.4 | 27.0 |
| eGFR Mean (mL/min) | 102 | 15.5 |

eGFR was calculated according to the Modification of Diet in Renal Disease (MDRD) equation (Levey A S et al., Clin Chem. Apr 2007;53(4):766-772).

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All documents cited herein are expressly incorporated by reference.

1. ZP1848 or a pharmaceutically acceptable salt thereof for use in prophylaxis or treatment of a condition responsive thereto in a subject with at least moderate renal impairment, wherein no dose adjustment of ZP1848 or said salt is required.

2. ZP1848 or a pharmaceutically acceptable salt thereof for use according to claim 1 wherein the subject has previously been treated with teduglutide at an adjusted dose.

3. ZP1848 or a pharmaceutically acceptable salt thereof for use in prophylaxis or treatment of a condition responsive thereto in a subject for whom an adjusted dose of teduglutide would be indicated as a result of renal impairment, wherein no dose adjustment of ZP1848 or said salt is required.

4. ZP1848 or a pharmaceutically acceptable salt thereof for use according to claim 3 wherein said subject has previously have been treated with teduglutide at an adjusted dose.

5. ZP1848 or a pharmaceutically acceptable salt thereof for use in prophylaxis or treatment of a condition responsive thereto in a subject who has received an adjusted dose of teduglutide due to renal impairment, wherein no dose adjustment of ZP1848 or said salt is required.

6. ZP1848 or a pharmaceutically acceptable salt thereof for use according to any one of claims 1 to 5 wherein the subject has moderate renal impairment, severe renal impairment, or end stage renal disease (ESRD).

7. ZP1848 or a pharmaceutically acceptable salt thereof for use according to any one of claims 1 to 6 wherein the condition to be treated is a stomach or bowel-related disorder.

8. ZP1848 or a pharmaceutically acceptable salt thereof for use according to claim 7 wherein said stomach or bowel related disorder is ulcers, a digestion disorder, a malabsorption syndrome, short-gut syndrome, inflammatory bowel disease, celiac sprue (for example arising from gluten induced enteropathy or celiac disease), tropical sprue, hypogammaglobulinemic sprue, enteritis, regional enteritis (Crohn's disease), ulcerative colitis, small intestine damage or short bowel syndrome (SBS).

9. ZP1848 or a pharmaceutically acceptable salt thereof for use according to claim 8 wherein the condition is short bowel syndrome (SBS) in a subject receiving parenteral support (PS).

10. ZP1848 or a pharmaceutically acceptable salt thereof for use according to claim 7 wherein the condition is radiation enteritis, infectious or post-infectious enteritis, or small intestinal damage due to toxic or other chemotherapeutic agents.

11. ZP1848 or a pharmaceutically acceptable salt thereof for use according to claim 7 wherein the condition is a side effect of chemotherapy or radiation treatment in a human subject.

12. ZP1848 or a pharmaceutically acceptable salt thereof for use in increasing intestinal mass and/or promoting or increasing longitudinal intestinal growth in a subject with at least moderate renal impairment, wherein no dose adjustment of ZP1848 or said salt is required.

13. ZP1848 or a pharmaceutically acceptable salt thereof for use according to claim 12 wherein the subject has previously been treated with teduglutide at an adjusted dose.

14. ZP1848 or a pharmaceutically acceptable salt thereof for use in increasing intestinal mass and/or promoting or increasing longitudinal intestinal growth in a subject for whom an adjusted dose of teduglutide would be indicated as a result of renal impairment, wherein no dose adjustment of ZP1848 or said salt is required.

15. ZP1848 or a pharmaceutically acceptable salt thereof for use according to claim 14 wherein the subject has previously been treated with teduglutide at an adjusted dose.

16. ZP1848 or a pharmaceutically acceptable salt thereof for use in increasing intestinal mass and/or promoting or increasing longitudinal intestinal growth in a subject who has received an adjusted dose of teduglutide due to renal impairment, wherein no dose adjustment of ZP1848 or said salt is required.

17. ZP1848 or a pharmaceutically acceptable salt thereof for use according to any one of claims 12 to 16 wherein the subject has moderate renal impairment, severe renal impairment, or ESRD.

18. ZP1848 or a pharmaceutically acceptable salt thereof for use according to any one of claims 12 to 17 wherein the subject is affected by short bowel syndrome (SBS) and optionally is receiving parenteral support.

19. ZP1848 or a pharmaceutically acceptable salt thereof for use according to any one of claims 1 to 18 wherein said ZP1848 or pharmaceutically acceptable salt is for administration via a dosing regime comprising a plurality or course of doses separated in time by 2 days, 2.5 days, 3 days, 3.5 days, 4 days, 5 days, 6 days, 7 days, 8 days, 9 days, 10 days, 11 days or 12 days, e.g. separated in time by 3 days, 3.5 days, 4 days, 5 days, 6 days, 7 days or 8 days, e.g. separated in time by 3 days, 3.5 days, 4 days or 7 days.

20. ZP1848 or a pharmaceutically acceptable salt thereof for use according to claim 19 wherein said dosing regime is a once or twice weekly dosing regime.

21. ZP1848 or a pharmaceutically acceptable salt thereof for use according to any one of claims 1 to 20 wherein each individual unadjusted dose is between 1 mg and 20 mg inclusive per subject per dose, e.g. between 5 mg and 15 mg, between 7 mg and 12 mg, or between 9 mg and 11 mg inclusive per subject per dose.

22. ZP1848 or a pharmaceutically acceptable salt thereof for use according to claim 21 wherein each individual unadjusted dose is about 10 mg per subject per dose.

23. A pharmaceutical kit comprising ZP1848 or a pharmaceutically acceptable salt thereof and information that no dose adjustment of ZP1848 is required for subjects having at least moderate renal impairment. 30

24. A kit according to claim 23 wherein said ZP1848 or pharmaceutically acceptable salt is provided as a pharmaceutical composition, comprising said ZP1848 or salt in combination with a pharmaceutically acceptable carrier or excipient.

25. A kit according to claim 23 or claim 24 comprising one or more individual measured doses of ZP1848 or said salt, wherein each said dose is between 1 mg and 20 mg, e.g. between 5 mg and 15 mg, between 7 mg and 12 mg, or between 9 mg and 11 mg.

26. A kit according to claim 25 wherein each individual dose is about 10 mg.

27. A kit according to any one of claims 23 to 26 further comprising one or more chemotherapeutic agents.
